(19) United States
(12) Patent Application Publication
(10) Pub. No.: US 2024/0358387 A1
(43) Pub. Date: Oct. 31, 2024

(54) THROMBECTOMY GUIDEWIRES AND METHODS FOR SAME

(71) Applicant: Surmodics, Inc., Eden Prairie, MN (US)

(72) Inventors: Derek Stratton, Bloomington, MN (US); Alex Marine, Eden Prairie, MN (US); Peter Bo, Eden Prairie, MN (US); Jordan Berg, Eden Prairie, MN (US)

(21) Appl. No.: 18/647,176

(22) Filed: Apr. 26, 2024

Related U.S. Application Data
(60) Provisional application No. 63/462,450, filed on Apr. 27, 2023.

(51) Int. Cl. A61B 17/221; A61B 17/00; A61B 17/22; A61M 25/09

(52) U.S. Cl. CPC A61B 17/221; A61B 2017/00336; A61B 2017/22044; A61B 2017/22072; A61B 2017/2212; A61M 2025/09183

(57) ABSTRACT

A thrombectomy guidewire system includes a thrombectomy guidewire having a wire shaft with a proximal portion and a distal portion, the wire shaft is sized for receiving and delivering guidewire deliverable intravascular thrombectomy devices. The thrombectomy guidewire includes a self-expanding basket positioned at the distal portion of the wire shaft and configured to self-expand from a collapsed configuration to an expanded configuration. A exchange sheath includes a first lumen configured to slidably receive the wire shaft and a second lumen aligned with the central lumen. The exchange sheath is slidable in a distal direction over the wire shaft from the proximal portion of the wire shaft at least until the self-expanding basket is sheathed within the central lumen and collapsed into the collapsed configuration.

### CLAIM OF PRIORITY

This patent application claims the benefit of priority, under 35 U.S.C. Section 119(e), to Stratton, et al., U.S. Provisional Patent Application Ser. No. 63/462,450, entitled “Thrombectomy Guidewires and Methods for Same,” filed on Apr. 27, 2023 (Attorney Docket No. 3028.075PRV), which is hereby incorporated by reference herein in its entirety.

### TECHNICAL FIELD

This document pertains generally, but not by way of limitation, to an intravascular system, and more particularly, is for an intravascular filter system for clot protection, removal, or maceration.

### BACKGROUND

Thrombectomy is a surgical procedure used to remove a clot from a vessel, such as an artery or vein. A thrombectomy procedure can target a clot such as a thrombus, which is a clot formed in situ within the blood vessel. Alternatively, thrombectomy procedures can also target blood clots such as embolisms, which are thrombus that have dislodged from the original formation site and have become relodged in a different region of the vascular system. A pulmonary embolism (“PE”) is a specific type of embolism that is dislodged from a formation site, often the lower leg, and has relodged in a blood vessel within the lungs. Each of these variations are referred to collectively herein as thrombus, clot or the like.

Mechanical thrombectomy procedures in some examples involve deployment of a capture device at the clot. The capture device is pulled through the clot to mechanically capture or entrain the clot for removal through a separate retrieval catheter or sheath.

Aspiration thrombectomy systems in other examples include placement of a catheter or sheath adjacent to the clot and drawing a vacuum (negative pressure) through the catheter to pull the clot material into the catheter and out of the body.

### SUMMARY OF THE INVENTION

Thrombectomy procedures typically involve removing thrombus from a blood vessel by aspirating the thrombus into a procedural sheath, mechanically pulling the thrombus into the procedural sheath, or combinations of both. Thrombus, as used herein, includes one or more of clot, particulate, thrombus or the like, and the terms may be used interchangeably.

A risk in thrombectomy procedures is embolization, where portions of thrombus separate from the main thrombus and become emboli traveling through the vasculature. Aspiration thrombectomy procedures involve applying suction to the thrombus through the procedural sheath to draw the thrombus material into the procedural sheath. Suction may cause the thrombus to break into fragments, that in some instances escape into the blood stream as emboli. Mechanical thrombectomy procedures involve deploying a thrombus engagement mechanism (e.g., balloon, basket) distal to the thrombus or midway within the thrombus to pull the thrombus material toward a procedural sheath. The thrombus engagement mechanism mobilizes the thrombus material and in some examples separates the thrombus material adhered to the vessel wall. The engagement of the thrombus engagement mechanism to the thrombus material may cause the thrombus material to break into fragments, that may escape into the blood stream. Likewise, larger or more organized thrombus fragments may overwhelm the thrombus engagement mechanism and escape past the thrombus engagement mechanism or aspiration through the procedural sheath and become emboli. The risk of embolization in some circumstances may discourage a physician from aggressively pursuing thrombus thereby leaving thrombus within a vessel or prompt the use of other thrombus treatments (i.e., thrombolytics) that have other drawbacks or side effects.

An approach to reducing embolization is deploying additional intravascular devices around the thrombus to isolate the thrombus and capture fragments or escaped thrombus. The additional intravascular devices are typically deployed through a sheath lumen of a procedural sheath, and the intravascular devices occupy some of the cross-sectional area of the sheath lumen. However, thrombus removed from the vessel is delivered through the sheath lumen of the procedural sheath, for instance by way of aspiration. Occupying or reducing the cross-sectional area of the sheath lumen (with intravascular devices) frustrates the extraction of thrombus through the procedural sheath and the thrombus may become stuck (e.g., seize) within the sheath lumen of the procedural sheath. Accordingly, addressing emboli with intravascular devices delivered through a sheath lumen may cause difficulty with extraction of captured thrombus in the procedural sheath.

Alternatively, certain thrombectomy systems have external features on the procedural sheath that are outside of the sheath lumen that are expandable to isolate the vessel. These exterior features in some examples are housed within additional lumens along the procedural sheath for controlling expansion of these exterior features. The inclusion of these exterior features, while outside of the sheath lumen, reduce the diameter of the sheath lumen thereby frustrating extraction of thrombus through the smaller sheath lumen.

An additional concern is the direction of flow past the thrombus. Depending on the particular position of the blockage and the relative orientation of the access site to the thrombus, the procedural sheath may be delivered upstream of the thrombus. In these situations, the disruption of the clot by the mechanical thrombectomy device may cause clot fragments to flow downstream. In certain situations, the clot fragments may then enter smaller downstream vessels that are too small to effectively or safely clear with mechanical thrombectomy devices.

The present inventors have recognized, among other things, that a problem to be solved can include decreasing the escape of mobile thrombus from thrombectomy procedures while at the same time ensuring captured thrombus is readily delivered through a procedural sheath for removal from a patient. The thrombectomy systems described herein are configured to conduct thrombectomy procedures and include a thrombectomy guidewire that acts as a rail for navigation and delivery of a thrombectomy system to a target location (e.g., thrombus within a vessel). For instance, a self-expanding basket of the thrombectomy guidewire locates the wire shaft of the thrombectomy guidewire centrally within the vessel and holds the wire shaft statically by way of engagement of the basket with the vessel wall. At the same time the self-expanding basket of the thrombectomy guidewire provides a net, screen or the like that captures mobilized thrombus that escapes from the thrombectomy system (e.g., mechanical mobilization, aspiration or the like).

Additionally, the thrombectomy guidewire is delivered through a catheter lumen of the thrombectomy system (already located in the procedural sheath) and accordingly does not further fill the sheath lumen of the procedural sheath. The thrombectomy guidewire thereby does not frustrate aspiration of thrombus through the procedural sheath.

This overview is intended to provide an overview of subject matter of the present patent application. It is not intended to provide an exclusive or exhaustive explanation of the invention. The detailed description is included to provide further information about the present patent application.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 is a side view of one example of a thrombectomy guidewire assembly including a self-expanding basket according to an embodiment of the present disclosure.

FIG. 2A is a side view of one example of a thrombectomy guidewire assembly including a self-expanding basket according to an embodiment of the present disclosure.

FIG. 2B is a detailed side view of the thrombectomy guidewire assembly of FIG. 1 including a self-expanding basket according to an embodiment of the present disclosure.

FIG. 3 is an end view of an example of the self-expanding basket of the thrombectomy guidewire assembly according to an embodiment of the present disclosure.

FIG. 4 is a partial side view of the self-expanding basket of FIG. 3 according to an embodiment of the present disclosure.

FIG. 5A is a side view of one example of a thrombectomy guidewire delivery system according to an embodiment of the present disclosure.

FIG. 5B is a top view of the thrombectomy guidewire delivery system of FIG. 5A according to an embodiment of the present disclosure.

FIG. 5C is a sectional side view of one example of the thrombectomy guidewire delivery system of FIG. 5A illustrating initial delivery of a sheathed self-expanding basket according to an embodiment of the present disclosure.

FIG. 5D is a detailed side view of the thrombectomy guidewire delivery system of FIG. 5A illustrating an unsheathed self-expanding basket according to an embodiment of the present disclosure.

FIG. 6A is a side view of a vessel including a clot.

FIG. 6B is a side view of a thrombectomy system delivered through a clot in a vessel according to an embodiment of the present disclosure.

FIG. 6C is a side view of the thrombectomy system of FIG. 6B including an unsheathed self-expanding basket distal to a clot in a vessel according to an embodiment of the present disclosure.

FIG. 6D is a side view of the thrombectomy system of FIG. 6B including an unsheathed self-expanding basket and an unsheathed expandable basket illustrated within a clot in a vessel according to an embodiment of the present disclosure.

FIG. 6E is an end view of the self-expanding basket of FIG. 6D according to embodiments of the present disclosure.

FIG. 6F is a side view of the thrombectomy system of FIG. 6B including two unsheathed expandable baskets within a clot and an unsheathed self-expanding basket distal to the clot in a vessel according to an embodiment of the present disclosure.

FIG. 7 is a side view of another example of a thrombectomy system including at least one self-expanding thrombus element according to an embodiment of the present disclosure.

FIG. 8A is a side view of a thrombectomy system according to an embodiment of the present invention with a delivery sheath in a fully sheathed position.

FIG. 8B is a side view of the thrombectomy system depicted in FIG. 7A with the delivery sheath positioned to unsheathe an adjustable basket.

FIG. 8C is a side view of the thrombectomy system depicted in FIG. 7A with the delivery sheath positioned to unsheathe the adjustable basket and a first debulking basket.

FIG. 8D is a side view of the thrombectomy system depicted in FIG. 7A with the delivery sheath positioned to unsheathe the adjustable basket, the first debulking basket, and a second debulking basket.

FIG. 9A is a side view of a thrombectomy system according to an embodiment of the present disclosure.

FIG. 9B is a partial cross-sectional side view of a thrombectomy system according to an embodiment of the present disclosure.

### DETAILED DESCRIPTION

During some example thrombectomy procedures, a procedural guidewire is navigated through the vasculature from an access site to the thrombus. The procedural guidewire is delivered across the thrombus to locate the distal end of the procedural guidewire distally relative to the thrombus. A procedural sheath is navigated to the thrombus from the access site over the procedural guidewire to position the distal end of the procedural sheath proximally relative to the thrombus. Intravascular devices are delivered through a sheath lumen of the procedural sheath to engage the thrombus and mobilize the thrombus into the sheath lumen for removal. Aspiration (e.g., suction) is optionally applied through the procedural sheath to extract the thrombus through a sheath lumen of the procedural sheath.

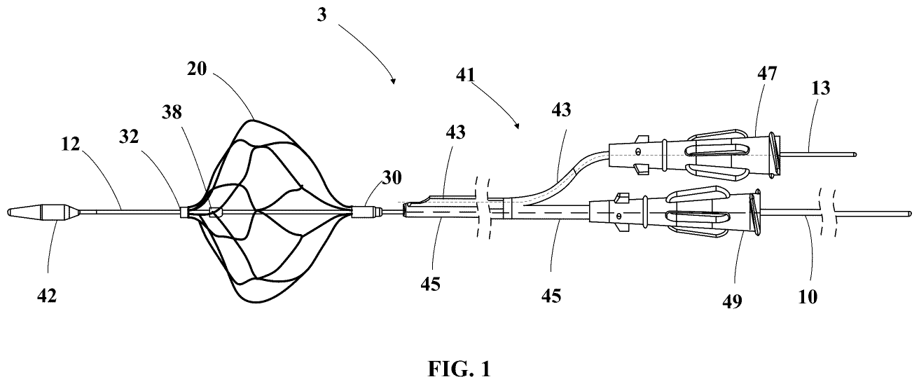

FIG. 1 is a side view of one example of a thrombectomy delivery system 3 according to an embodiment of the present disclosure. The thrombectomy delivery system 3 includes a thrombectomy guidewire 10 having a wire shaft 12 and a self-expanding basket 20 coupled to the thrombectomy guidewire 10. The thrombectomy delivery system 3 further includes an exchange sheath 41 that facilitates the exchange of a procedural guidewire 13 with the thrombectomy guidewire 10, as described herein. In the example shown in FIG. 1, the exchange sheath 41 includes a guidewire insertion portion 49 that is connected to a first lumen 45. The thrombectomy guidewire 10 is inserted through the guidewire insertion portion 49 and the first lumen 45 (the thrombectomy guidewire 10 within the first lumen 45 is illustrated by the dashed line) of the exchange sheath 41. The guidewire insertion portion 49 includes a tapered fitting such that the thrombectomy guidewire 10 is insertable and guided toward and into the first lumen 45. The exchange sheath 41 of the thrombectomy delivery system 3 includes a procedural insertion portion 47 that includes a tapered fitting that guides an element, such as a procedural guidewire 13, toward a second lumen 43. The procedural guidewire 13 within the second lumen 43 is illustrated by the shorter dashed line in comparison to the longer dashes of the thrombectomy guidewire 10). The first lumen 45 and the second lumen 43 are, in one example, coupled along a distal portion and are split at a proximal portion. As shown, the first lumen 45 and the second lumen 43 split into the procedural insertion portion 47 and the guidewire insertion portion 49.

The tapered fitting of the procedural insertion portion 47 facilitates introduction and retraction of a procedural guidewire 13 to or through the second lumen 43 and ultimately location of the procedural guidewire 13 into a vessel. Similarly, the tapered fitting of the guidewire insertion portion 49 permits introduction and retraction of the thrombectomy guidewire 10 (or guidewire) to or through the first lumen 45 and ultimately location of the thrombectomy guidewire 10 into a vessel.

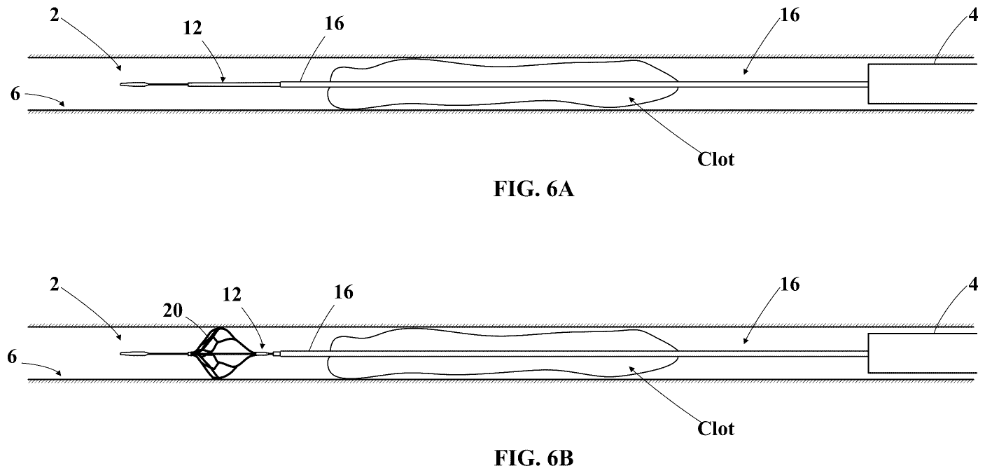

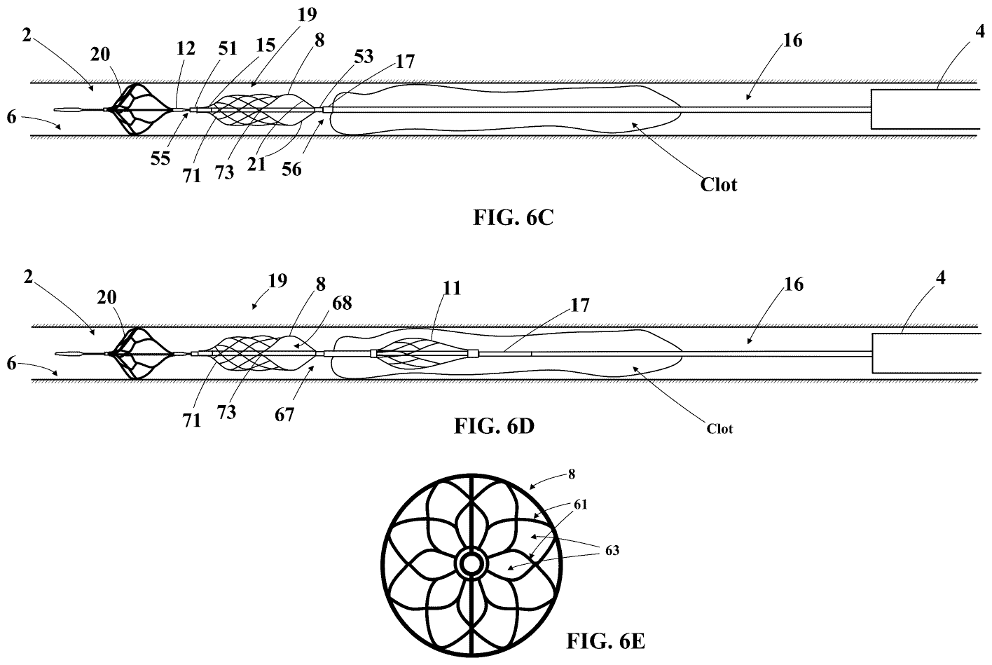

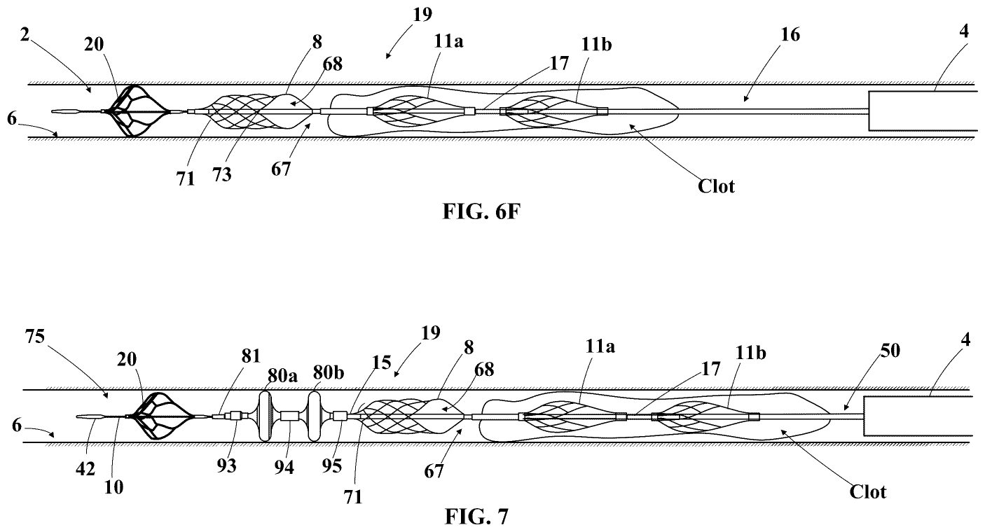

In one example, the procedural guidewire 13 is initially placed in the vessel. The exchange sheath 41 permits the delivery of the thrombectomy guidewire 10 along the procedural guidewire 13 to a target location to permit locating of the thrombectomy guidewire 10 at the target location (including proximate to the target location). For example, the exchange sheath 41 of the thrombectomy delivery system 3 permits the exchange of the procedural guidewire 13 for the thrombectomy guidewire 10. With the thrombectomy guidewire 10 deployed (as shown in FIG. 1) the self-expanding balloon 20 anchors the wire shaft 12 in place and centered to correspondingly center conduct of a thrombectomy procedure within a vessel. Additionally, the self-expanding basket 20 intercepts and arrests errant thrombus particulate that may escape the proximal thrombectomy device (e.g., the adjustable basket 8, or self-expanding thrombus elements 11a, 11b shown in FIGS. 6C to 9B). Once the thrombectomy guidewire 10 is placed within the vessel (e.g., exchanged with the procedural guidewire 13), the procedural guidewire 13 is retracted through the exchange sheath 41 and the thrombectomy guidewire 10 serves as the procedural guidewire.

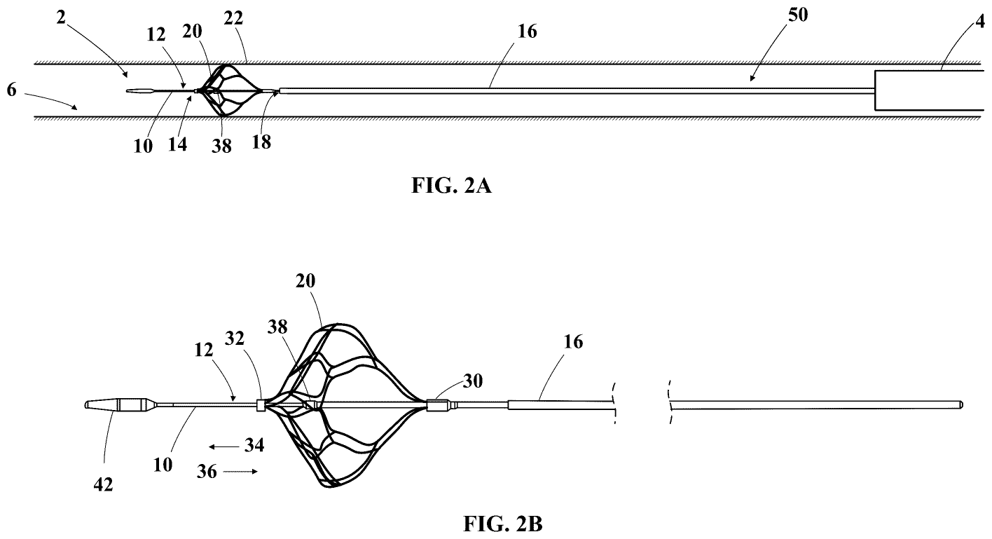

In the expanded configuration, the self-expanding basket 20 spans a vessel (such as vessel 6 in FIG. 2A) and its fenestrated structure permits blood flow while capturing mobilized thrombus, thereby reducing embolization risk. Further, the self-expanding basket 20 engages with the vessel wall 22 to maintain the position of the self-expanding basket 20 and the wire shaft 12 coupled with the basket 20 within the vessel 6. For instance, the self-expanding basket 20 positions the wire shaft 12 within a central portion of the vessel 6, and also axially maintains its position along the vessel 6 by way of engagement with the vessel wall 22. The wire shaft 12, coupled with the self-expanding basket 20, in the deployed configuration, provides a rail for delivery of thrombectomy devices to a treatment location (e.g., thrombus), as shown in the examples of FIGS. 6A-6F.

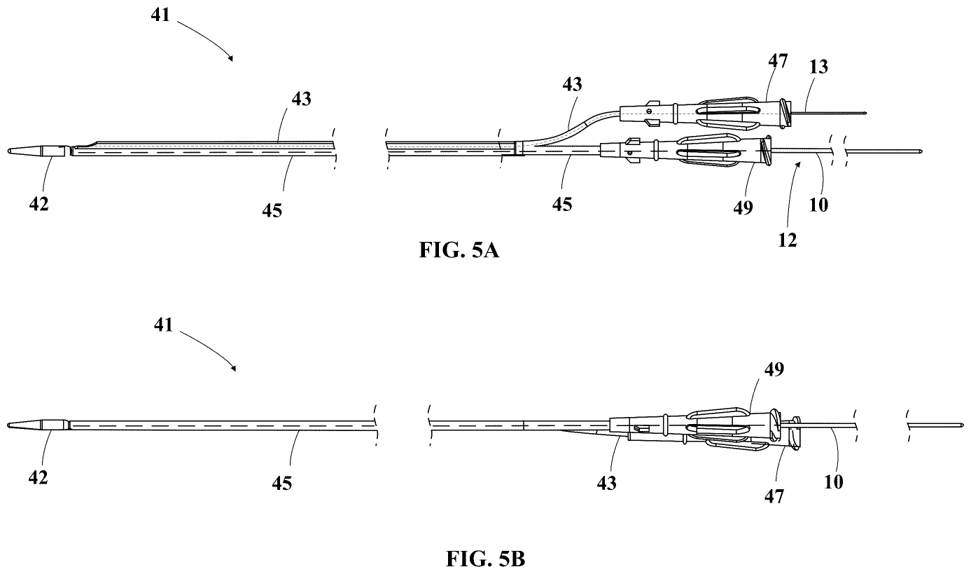

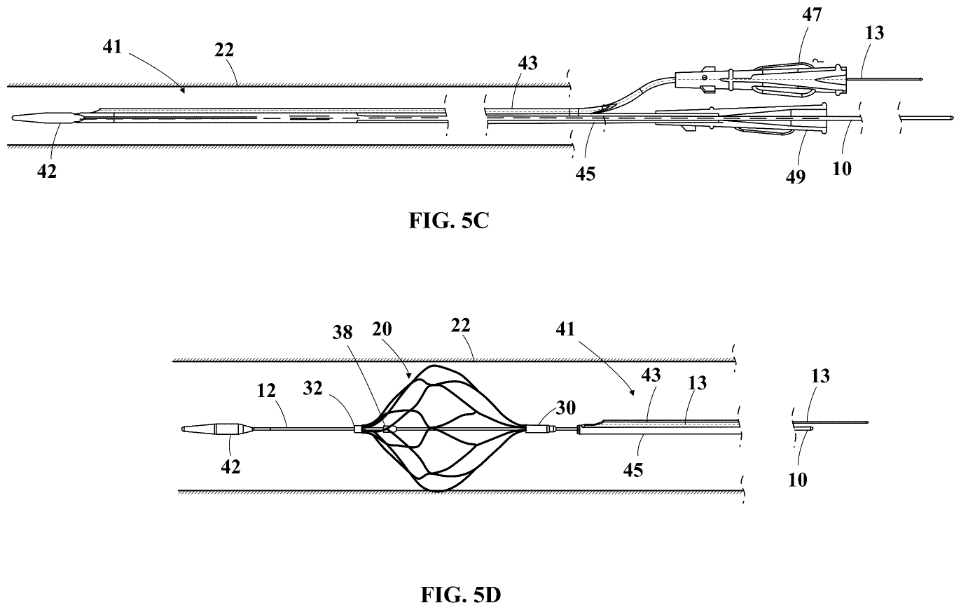

FIG. 2A illustrates one example of a thrombectomy system 2 including a thrombectomy guidewire 10 having a wire shaft 12 and a self-expanding basket 20 positioned at a distal portion 14 of the wire shaft 12. The thrombectomy guidewire 10 includes, in an example, a guidewire sheath 16 having a sheath lumen 18 (sometimes referred to as a central lumen) configured to slidably receive the wire shaft 12. The guidewire sheath 16 is slidable over the wire shaft 12 (distally) to stow (e.g., sheath, collapse, compress or the like) and constrain the self-expanding basket 20 within the sheath lumen 18 in a collapsed configuration. Optionally, the wire shaft 12 is retracted proximally and collapsed by the guidewire sheath 16. The guidewire sheath 16 is also slidable in a proximal direction to deploy (e.g., unsheathe, expand, reveal or the like) the self-expanding basket 20. The self-expanding basket 20 includes one or more materials, such as shape memory materials or the like (e.g., Nitinol®, piano wire) that are configured to self-expand from the collapsed configuration to an expanded configuration (e.g., stowed to deployed configurations) with the basket unconstrained. The guidewire sheath 16 is one embodiment used to place the thrombectomy guidewire. Another embodiment, including using the exchange sheath 41 shown in FIG. 1, is described further below in association with FIGS. 5A-5D.

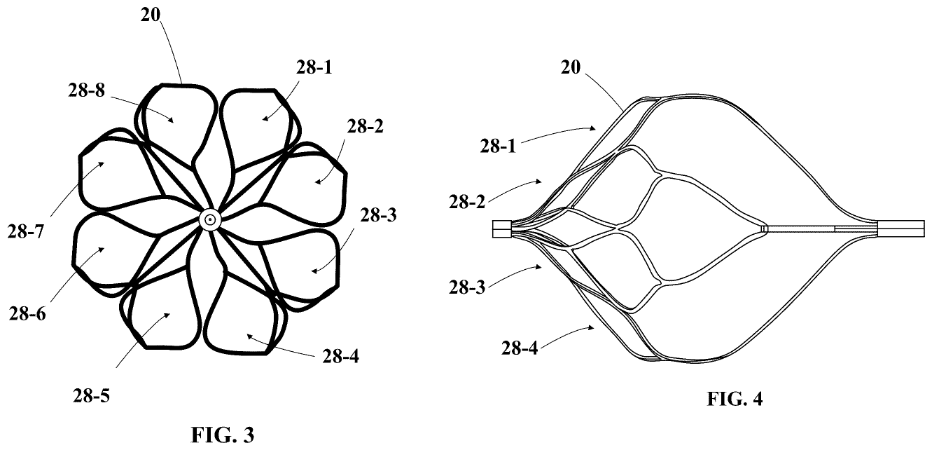

FIG. 2B is a detailed view of the self-expanding basket 20 previously shown in FIG. 2A. The self-expanding basket 20 includes a fenestrated structure (e.g., struts, wires, filaments, mesh or the like) extending between a proximal collar 30 and a distal collar 32. The fenestrated structure, in one example, includes a plurality of intervening openings, also shown in an end view of FIG. 3 with a plurality of intervening openings 28-1 to 28-8. The enlarged side view of the fenestrated structure in FIG. 4 also shows the openings 28-1, 28-2, 28-3, 28-4, permitting the flow of fluid through the fenestrated structure while thrombus material is engaged by the fenestrated structure. As shown in FIG. 2B, the proximal collar 30 is anchored to the wire shaft 12 and the distal collar 32 is slidably attached to the wire shaft 12, in one example. In operation, the distal collar 32 is moveable in the distal direction 34 to collapse the fenestrated structure into the collapsed configuration against the wire shaft 12, for instance as the basket 20 is retracted into a sheath. Similarly, the distal collar 32 is moveable in the proximal direction 36 to permit the fenestrated structure to expand radially away from the wire shaft 12 and span the vessel 6 (occlude while also permitting blood flow) to capture thrombus fragments moving distally, for instance from thrombectomy devices proximal to the basket 20.

In at least one embodiment, the wire shaft 12 includes a wire stop 38 that limits travel of the distal collar 32 in the proximal direction 36. The wire stop 38 arrests proximal movement and limits the corresponding radial expansion of the self-expanding basket at a predetermined outer diameter. In one example, thrombus material fills the fenestrated structure, and excessive thrombus material may cause the self-expanding basket 20 to overly expand radially and potentially evert. The wire stop 38 mechanically limits overwhelming of the self-expanding basket 20 by thrombus material and increases the quantity of thrombus material captured by the self-expanding basket 20 (relative to a basket without the wire stop 38) while decreasing the chance of an eversion.

In another embodiment, in the expanded configuration, the self-expanding basket 20 is spans a vessel 6 (e.g., occludes while permitting blood flow and capturing thrombus), including large diameter vessels such as the vena cava, having diameters up to 25 mm. In at least one embodiment, the self-expanding basket 20 is sized to expand to an outer diameter greater than the inner diameter of the vessel such that the self-expanding basket 20 expands until constrained by the vessel and applies outward radial force against the vessel wall 22 to hold the basket 20 and wire shaft 12 in place while limiting damage (including no damage) to the vessel wall. In certain embodiments, the self-expanding basket 20 has an outer diameter of at least 30 mm when in a deployed configuration unconstrained by a vessel wall. In other embodiments, the outer diameter of the self-expanding basket 20 is about thirty-two (32) mm to about thirty-six (36) mm when in the deployed configuration (and unconstrained). The outward radial force applied by the self-expanding basket 20 against the vessel wall 22 intimately engages the self-expanding basket 20 with the wall 22 and maintains the position of the self-expanding basket 20, and correspondingly the wire shaft 12, within the vessel 6. The self-expanding basket 20, in an example, positions the wire shaft 12 in the approximate center of the vessel 6 and does so while limiting damage (e.g., no damage, minimal abrasion or the like) to the vessel wall.

Referring now to FIGS. 5A-5C, an exchange sheath 41 is delivered over a procedural guidewire 13 and the procedural guidewire 13 is extracted through the exchange sheath 41. For example, the procedural guidewire 13 is initially placed in a vessel and the exchange sheath 41 tracks along the procedural guidewire 13 for insertion into the vessel. Prior to delivery of the exchange sheath 41 over the procedural guidewire 13, a thrombectomy guidewire 10 is inserted into a first lumen 45 of the exchange sheath 41, starting with a proximal portion of the thrombectomy guidewire 10 being inserted into a distal portion of the exchange sheath 41, where a wire tip 42 positions the distal portion of the thrombectomy guidewire 10. In some examples, the procedural guidewire 13 is removed after the exchange sheath 41 is placed in the vessel. Further, in some alternative examples where the wire tip 42 is small enough, once the exchange sheath 41 is in place, the thrombectomy guidewire 10 is inserted through the lumen 45 of the exchange sheath 41, to be placed in the vessel. Upon placement of the thrombectomy guidewire 10 in the vessel, the exchange sheath 41 is also removed.

As an example method, the exchange sheath 41 is advanced over the wire shaft 12 of a thrombectomy guidewire 10, where the wire shaft 12 is received within a first lumen 45 of the exchange sheath 41. The exchange sheath 41 is slid distally over a self-expanding basket 20 on a distal end of the wire shaft 12 to collapse the self-expanding basket 20 to a collapsed configuration. As mentioned above, a procedural guidewire 13 is navigated through a vessel 22 containing a thrombus such that a distal end of the procedural guidewire 13 is positioned distal to a clot. The exchange sheath 41 is advanced with the thrombectomy guidewire 10 over the procedural guidewire 13, received within a secondary lumen 43 of the exchange sheath 41, until the self-expanding basket 20 (within the exchange sheath 41) is positioned distal to the clot. With the thrombectomy guidewire 10 at a target location the procedural guidewire 13 is withdrawn from the vessel 22 through the secondary lumen 43 of the exchange sheath 41. The exchange sheath 41 is withdrawn over the wire shaft 12 of the thrombectomy guidewire 10 to unsheathe the self-expanding basket 20 and permit expansion of the self-expanding basket 20 within the vessel 22 distal to the clot.

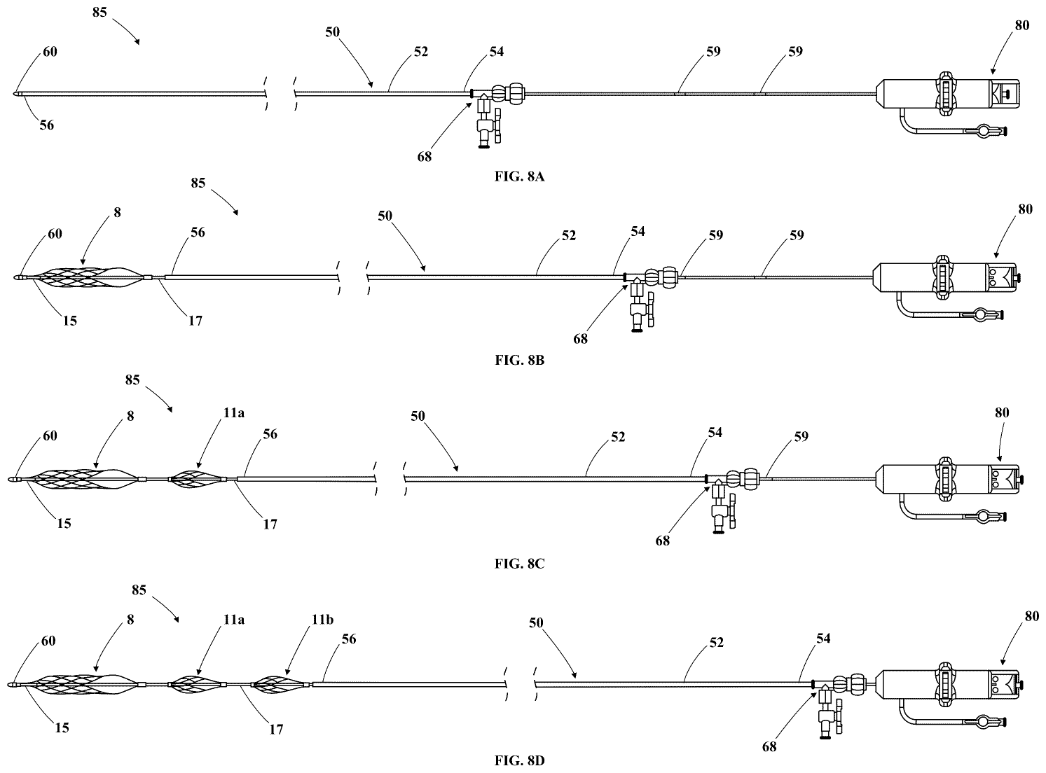

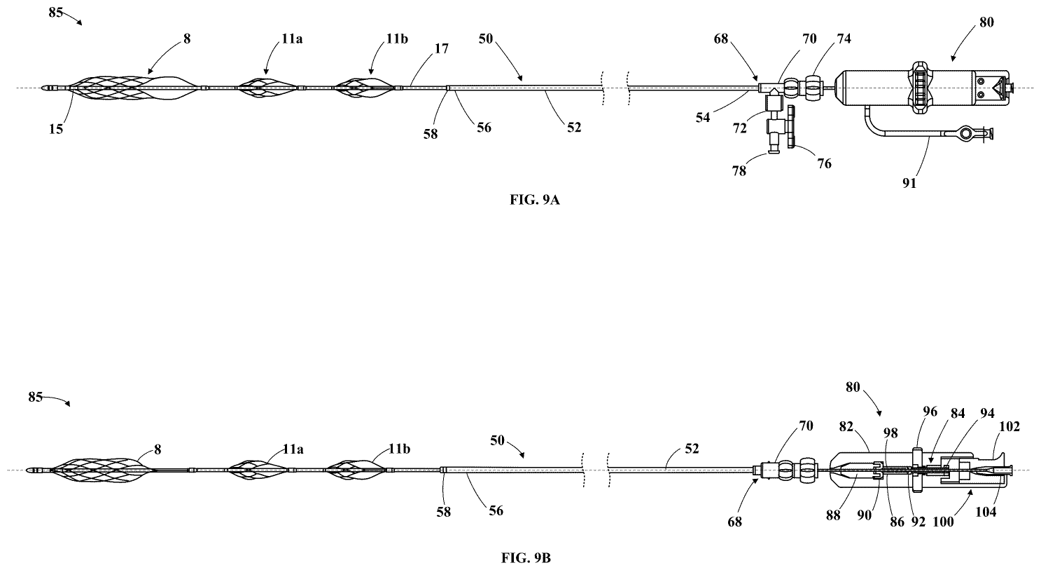

In another example, a procedural sheath may be advanced over the procedural guidewire 13 such that a proximate end of the procedural sheath is proximate to the clot. An intravascular device (such as those shown in FIGS. 8A to 9B) is advanced over the thrombectomy guidewire 10 (having the wire shaft 12) to the clot. For example, the intravascular device is navigated over the thrombectomy guidewire 10 while within the procedural sheath. In other words, the guidewire 10 is nested within the intravascular device, and the intravascular device is nested within the procedural sheath.

The procedural sheath is retracted to permit deployment of one or more features of the intravascular devices, such as, but not limited to, baskets, cages, aspiration ports, fluid-mechanical or fluid features. The clot is engaged with one or more of these features of the intravascular device. The exchange sheath 41 is slid distally over an adjustable basket (e.g., basket 8 shown in FIGS. 8A to 8D) and a sheath. The sheath is slidably coupled along the wire shaft 12 and over self-expanding thrombus elements (e.g., such as self-expanding thrombus elements 11a, 11b in FIGS. 8A to 8D) slidably coupled along the wires shaft 12 and proximal of the adjustable basket. The exchange sheath 41 is withdrawn over the wire shaft 12 to unsheathe the adjustable basket and the self-expanding thrombus elements. Referring again, to the self-expanding basket 20 of the thrombectomy guidewire 10, the basket 20 captures emboli potentially separated from the clot during engagement of the intravascular device. Stated another way, the self-expanding basket 20 intercepts separated clot that may otherwise move downstream from the treatment location.

Since, in at least one embodiment, the wire shaft 12 of the thrombectomy guidewire 10 has an outer diameter compatible with other intravascular devices, other thrombectomy elements use the wire shaft 12 for delivery (in the manner of a rail) even with the procedural guidewire 13 extracted. The wire shaft 12 includes an outer diameter of at least about 0.014 inches, about 0.018 inches, about 0.035 inches or the like. In this configuration, the wire shaft 12 provides a rail for delivery of other intravascular devices to the thrombus in place of the procedural guidewire 13. In at least one embodiment, the wire shaft 12 can be sized to fit within the guide wire sheath 16, wherein the guide wire sheath 16 can have an outer diameter of at least about 0.014 inches, about 0.018 inches, about 0.035 inches or the like. In this configuration, the wire sheath 16 can be partially withdrawn to allow the self-expanding basket 20 while remaining over the majority of the wire shaft 12 to act as a rail for delivery of other intravascular devices to the thrombus in place of the procedural guidewire 13. In certain embodiments, at least one of the wire shaft 12, wire sheath 16, and combinations thereof can be coated with a hydrophilic lubricious coating.

In operation, the thrombectomy guidewire 10 is, in one example, delivered across the thrombus via the procedural guidewire 13. In one embodiment, an exchange sheath 41 includes a first (e.g., a thrombectomy guidewire delivery) lumen 45 and a second (e.g., a procedural guidewire exchange) lumen 43. In some examples, the second lumen 43 receives the procedural guidewire 13 through a procedural insertion portion 47, allowing the thrombectomy guidewire 10 to navigate across thrombus via the procedural guidewire 13.

As shown in FIGS. 5A, C, a thrombectomy wire tip 42 of the thrombectomy wire 10 sockets into the exchange sheath 41, for instance into the first lumen 45, for an atraumatic transition. In some examples, the wire tip 42 is tapered and positioned on the distal end of the wire shaft 12. The wire tip 42 tapers radially from a larger diameter proximal portion of to a smaller diameter distal portion of the wire tip 42. The profile of the proximal end of the wire tip 42 approximates the profile of the exchange sheath 41 (e.g., the combined profiles of the walls surrounding the lumens 43, 45). The exchange sheath 41 is slidable over the wire shaft 12 in the distal direction (e.g., backloaded) to abut the distal end of the exchange sheath 41 against the proximal end of the wire tip 42.

Upon positioning of the sheathed self-expanding basket 20 distal to the thrombus the exchange sheath 41 is retracted relative to the thrombectomy guidewire 10 thereby permitting the self-expanding basket 20 to expand and anchor within the vessel 6 (e.g., in the expanded or deployed configuration), as illustrated in FIG. 5D. The self-expanding basket 20, when deployed, is a distal brace that eases navigation of intravascular devices in the distal direction 34 over the wire shaft 12 to the thrombus while reducing the likelihood the thrombectomy guidewire shifts axially (e.g., distally or proximally along the vessel). In addition, the self-expanding basket 20 aligns the wire shaft 12 proximate to the center of the vessel 6, further easing navigation of intravascular devices over the wire shaft 12 as well as centering the devices and improving the effectiveness of the intravascular devices in engaging and removing thrombus through a centralized deployment in contrast to deployment proximate to a vessel wall.

Referring now in FIG. 6A, another example of a thrombectomy system 2 (an example of an intravascular thrombectomy device), according to an embodiment of the present subject matter, includes a self-expandable basket (such as self-expandable basket 20 in FIGS. 6B-6D, sheathed in FIG. 6A), that is distal to a clot (or thrombus) in a vessel 6. The sheathed self-expandable basket is within a procedural sheath 16 over a wire shaft 12. As shown in FIG. 6B, the self-expandable basket 20 is unsheathed and expanded radially in close proximity to the vessel walls.

As shown in FIG. 6C, an intravascular catheter assembly 19 including an adjustable basket 8 is deployed distal to the clot. As shown herein, the adjustable basket 8 and potentially other features on the intravascular catheter assembly 19 are pulled proximally to engage and mobilize the clot toward a procedural sheath 16 positioned proximal to the clot. The adjustable basket 8 includes a fenestrated structure extending between a distal collar 51 attached to a distal end 55 of the inner catheter 15 and a proximal collar 53 attached to a distal end of the outer catheter 17. The inner catheter 15 is slidable in the distal direction with respect to the outer catheter 17 to radially collapse (e.g., compress) the fenestrated structure against the inner catheter 15. The inner catheter 15 is likewise slidable in the proximal direction with respect to the outer catheter 17 to radially expand (e.g., dilate, open or the like) the fenestrated structure to generally occlude the vessel 6 containing the clot or thrombus. In an example, occlusion includes spanning the vessel 6 and filling the vessel with the fenestrated structure to mobilize and capture thrombus while permitting blood flow through the fenestrated structure. The outer diameter 17 of the adjustable basket 8 is in one example manually adjusted by sliding the inner catheter 15 allowing the outer edge of the adjustable basket 8 to skim or scrape the vessel wall.

In operation, the inner catheter 15 is slid proximally relative to the outer catheter 17 to expand the adjustable basket 8 to approximate the inner diameter vessel. The inner catheter 15 is optionally further slid proximally to “over-expand” the adjustable basket 8 toward the vessel wall to provide intimate engagement therebetween and apply a radial force against the vessel wall. The over-expansion of the adjustable basket 8 aids in scraping and separation of wall adherent thrombus material from the vessel wall. The adjustable basket 8 is deformable and complies with variations of profile along the vessel wall (e.g., diameter, contour, angles, bends or the like). Accordingly, the over-expanded adjustable basket 8 readily engages with thrombus adhered to the vessel wall, mobilizes the thrombus, and captures the thrombus within the adjustable basket 8 optionally while conducting one or a relatively small number of passes in comparison to other devices smaller or less intimately engaged to the vessel wall that fail to mobilize and capture thrombus as well as the intravascular catheter assembly 19 having the basket 8.

In certain embodiments, as shown from the end view of the adjustable basket 8 in FIG. 6E, the fenestrated structure of the adjustable basket 8 includes a plurality of struts 61 defining a plurality of flow openings 63 permitting fluid to flow through the fenestrated structure while thrombus material is engaged by the struts. Referring now to FIG. 6C, the fenestrated structure includes a closed distal end having the struts (such as struts 61 in FIG. 6E) taper to a distal collar 51 fixed to a distal end of the inner catheter 15. The fenestrated structure further includes a proximal end 67 (FIG. 6D), wherein the struts 61 (FIG. 6E) surround an open mouth 68. The proximal end 67 of the fenestrated structure is coupled to a proximal collar 53 fixed to a distal end 56 of the outer catheter 17 by one or more legs 21 (shown in FIG. 6C, in the present example, as two legs). The legs 21 are attached to opposed sides (such as, but not limited to, opposite) of the lip of the mouth 68 defined by the proximal end of the fenestrated structure. The adjustable basket 8 including the mouth 68 is expanded (e.g., made larger) with proximal movement of the inner catheter 15 relative to the outer catheter 17. Pulling of the expanded mouth 68 proximally toward the thrombus, mobilizes and directs the thrombus material through the open mouth 68 where it is trapped within the closed distal end 71.

After initial expansion of the adjustable basket 8 to an expanded diameter, the inner catheter 15 is slid further proximally relative to the outer catheter 17, which causes the legs (also referred to as a basket jack) to bow outward and ‘jack’ open the basket mouth 68 to enlarge the mouth 68 of the fenestrated structure. In at least one embodiment, the lip of the mouth 68 includes one or more notches or recesses each positioned between the legs. The notches are joints between opposed portions of the mouth and permit widening of the mouth 68 as the legs 21 drive the mouth 68 open and outward to enlarge the mouth 68 of the fenestrated structure, for instance to intimately engage along a vessel wall. The enlarged mouth opening 68 enhances intimate engagement with the vessel walls and increases the thrombus size that readily enters and is captured by the fenestrated structure. The legs 21, in another example, also include a generally rectangular cross-section having a narrow profile portion of the legs 21 that is directed outwardly toward the vessel walls when the legs are bowed (e.g., the mouth 68 is enlarged) and the portion of the legs 21 that bends towards 53 is directed proximally. These legs 21 act as cutting surfaces for further macerating large portions of thrombus material to ease receipt of the thrombus material through the mouth 68 and into the adjustable basket 8.

As shown in FIG. 6D, in an embodiment, the thrombectomy system 2 can further include at least one self-expanding thrombus element 10 positioned on the outer catheter 17 proximate to the adjustable basket 8. Each self-expanding thrombus element 10 can include a fenestrated structure, a braided structure, mesh, filaments, struts or combinations thereof to permit fluid flow through the self-expanding thrombus element 10 while engaging thrombus material. The self-expanding thrombus element 10 expands from a collapsed configuration to an expanded configuration when deployed, for instance with retraction of a procedural sheath 16 (also referred to as unsheathing of the self-expanding thrombus element). The thrombectomy system 2 can further include a thrombectomy sheath 16 defining a sheath lumen for slidably receiving the one or more self-expanding thrombus elements 10, adjustable basket 8 or the like. The thrombectomy sheath 16 (e.g., a procedural sheath, dedicated thrombectomy sheath or the like) can move distally to sheath the one or more self-expanding thrombus elements 10, adjustable basket 8 or the like, and thereby constrain and collapse each self-expanding thrombus element 10 and the adjustable basket 8. During deployment, the thrombectomy sheath 16 can be slid proximally to selectively unsheathe any number of self-expanding thrombus elements 10, the adjustable basket 8 or the like according to the size of the thrombus or other needs.

The self-expanding thrombus elements 10 are positioned on the outer catheter 17 a first predetermined distance proximal to the adjustable basket 8 such that the self-expanding thrombus elements 10 engage and mobilize a segment of the thrombus material separately from the adjustable basket 8. The self-expanding thrombus elements 10 can also macerate thrombus material into small fragments that are more easily collected by the trailing adjustable basket 8. Likewise, adjacent self-expanding thrombus elements 11a, 11b, as shown in FIG. 6F are separated a second predetermined distance such that each adjacent self-expanding element 10 engages a separate portion of the thrombus material. The spacing between the adjustable basket 8 and adjacent self-expanding thrombus elements 11a, 11b effectively divides the thrombus into smaller portions allowing each element to separately engage and mobilize a smaller mass of thrombus. Moreover, the smaller portions of thrombus are readily received in the procedural sheath 16 (e.g., for aspiration), and compression of otherwise large thrombus portions into plugs is decreased. Accordingly, the smaller portions of thrombus are less likely to compress into thrombus plugs that frustrate extraction or seize within the procedural sheath 16.

In certain embodiments, the thrombectomy system 2 is deployed at a thrombus with the adjustable basket 8 distal to the thrombus and each self-expanding thrombus element 11a, 11b deployed within the thrombus material. In this arrangement, long and organized thrombus is divided between the adjustable baskets 8 and self-expanding thrombus elements 11, 11b. Further, as the self-expanding thrombus elements 11a, 11b are deployed within the thrombus and the adjustable basket 8 is deployed distal to the thrombus, the procedural guidewire 13 extends just past the thrombus to support the adjustable basket 8. Accordingly, the thrombectomy system 2 captures and removes long and organized thrombus with a relatively short runway thereby shortening deployment of thrombus engagement elements within healthy or un-occluded vessel or organs and scraping of the thrombus engagement elements along those healthy vessels or organs. Further, as the thrombectomy system is extracted through the procedural sheath 16, the self-expanding basket 20 can prevent portions of the thrombus from escaping the thrombectomy system 2 and traveling through other portions of the vessel 6 distal to the self-expanding basket 20. In this way, loose material from the thrombus not collected by the adjustable basket 8 or the self-expanding thrombus elements 11a, 11b can be collected and extracted by the self-expanding basket 20.

As shown in FIG. 7, in an embodiment, another example of the thrombectomy system 75 includes at least one brush element 80a, 80b coupled with the inner catheter 15 distal to the adjustable basket 8. The brush element 80a, 80b (two are provided in this example) includes a fenestrated structure (e.g., a braid, mesh, filaments, struts or the like, or combinations thereof) that self-expands to form at least one radially projecting disc. In certain embodiments, the thrombectomy system 75 includes a brush catheter 81 slidably receivable within the inner catheter 15. In this configuration, the brush element(s) 80a, 80b include a fenestrated structure with a distal end of the fenestrated structure attached to the brush catheter 81 and a proximal end of the fenestrated structure attached to the inner catheter 15. The brush catheter 81 is optionally moved proximally relative to the inner catheter 15 to draw the ends of the fenestrated structure together and expand a portion of the fenestrated structure radially outward into one or more radially projecting discs.

As described above, the thrombectomy system 75 includes at least two expandable brush elements 80a, 80b positioned along the outer catheter 17. The at least two expandable brush elements 80a, 80b are coupled with a distal collar 93 and a proximal collar 95. The proximal collar 95 is attached to a distal portion of the outer catheter 17. The distal collar 93 is attached to a distal portion of the inner catheter 15. Generally, as shown in FIG. 7, the inner catheter 15 is moved proximally relative to the outer catheter 17 thereby reducing the distance between the proximal collar 95 and the distal collar 93. The movement longitudinally compresses and radially expands the brush elements 80a, 80b. Conversely, as the inner catheter 15 is moved in a distal direction relative to the outer catheter 17, the distance between the proximal collar 95 and the distal collar 93 is increased, thereby longitudinally expanding and radially contracting the brush elements 80a, 80b.

In some embodiments, the expandable brush elements 80a, 80b are constructed with a piece of mesh (e.g., optionally a continuous woven mesh). An intermediate collar 94 is clamped (e.g., pinched, gathered, anchored, or crimped) at a middle portion of the expandable brush elements 80a, 80b. In these embodiments, the two expandable brush elements 80a, 80b of the mesh are radially expanded together. A proximal collar 95 clamps the proximal expandable brush element 80b with the outer catheter 17. In another example, a distal collar 93 clamps the distal expandable brush element 80a with the inner catheter 15. In this way, proximal movement of the inner catheter 15, for instance telescoping proximal movement relative to the outer catheter 17, causes the distal collar 95 to move toward the proximal collar 93 and the brush elements 80a, 80b, to radially expand. Conversely, the collars maintain the mesh portions proximate to the collars (and optionally underlying the collars) to remain compressed.

In operation, as shown in FIG. 7, the brush element(s) 80a, 80b is in one example deployed by pulling the brush catheter 81 proximally relative to the inner catheter 15 to form at least one radially projecting disc. Optionally, the brush elements 80a, 80b self-expand when deployed, for instance according to construction with shape memory materials, such as nickel-titanium (also referred to as Nitinol®), piano wire or the like. The brush catheter 81 and inner catheter 15 are optionally selectively locked together (e.g., with a clamp, collet or the like) to prevent relative movement of the brush catheter 81 and inner catheter 15 thereby maintaining the brush element 80a, 80b in a deployed configuration. The brush catheter 81 and inner catheter 15 (in the locked configuration) are moved together to move the one or more radially projecting discs of the brush element 80a, 80b proximally, distally, or both (reciprocally) within the vessel. The one or more discs including one or more of the edge of the fenestrated structure, interior or the like are used to scour the vessel wall to free wall adherent thrombus. In particular, the discs are moved in reciprocating, rotating, proximal and distal motions (e.g., with varying reciprocation lengths, such as 1 millimeter, 5 millimeters, 1 centimeter or the like) or both to locally abrade adhered thrombus.

Optionally, as shown in FIG. 7, the radially projecting discs remain deployed as the thrombectomy brush element 80a, 80b is pulled through the procedural sheath 16. As the brush element 80a, 80b is pulled through the procedural sheath 16, the discs gather smaller fragments that escape the adjustable basket 8 or free thrombus adhered to the interior wall of the procedural sheath 16 (e.g., particulate). The thrombus particulate freed by the discs is optionally aspirated through the procedural sheath 16 or mechanically moved by the brush element 80a, 80b into the sheath 16. Further, as the thrombectomy system 75 is extracted through the procedural sheath 16, the self-expanding basket 20 (of the thrombectomy guidewire 10) prevents thrombus from escaping the thrombectomy system 75 and traveling through other portions of the vessel 6 distal to the self-expanding basket 20. In this way, loose thrombus not collected by the adjustable basket 8, the self-expanding thrombus elements 11a, 11b, or the brush element(s) 80a, 80b is collected and extracted by the self-expanding basket 20. Accordingly, a multi-tiered collection of features provide a composite mechanism for capture of thrombus for the thrombectomy system 75.

In certain embodiments, the one or more radially projecting discs have an outer diameter or profile (between about 8 mm to 16 mm) that differs or (in another example) matches the diameter or profile of the expanded adjustable basket 8 (14 mm to 18 mm). The varied diameters of the discs and the adjustable basket 8 permit the thrombectomy system 2 to readily treat different regions of vessels having different profiles (e.g., diameters, shapes, bends, angles or the like). Similarly, the fenestrated structure of the brush element(s) 80a, 80b optionally includes a plurality of stacked radially projecting disks with one or more of the disks having a different profile relative other disks in the stack.

As shown in FIGS. 8A to 9B, another example of a thrombectomy system 85 includes a delivery sheath 50 slidable over the intravascular catheter assembly 19. The delivery sheath 50 includes an elongated sheath body 52 having a proximal end 54 and a distal end 56 with a lumen extending between the proximal end 54 and the distal end 56. The intravascular catheter assembly 19 is slidably received within the delivery sheath 50 wherein the delivery sheath 50 is slidable axially over the intravascular catheter assembly 19 to sheath and unsheathe the adjustable basket 8 and the self-expanding thrombus elements 11a, 11b. The distal port 56 of the sheath body 52 is optionally reinforced to prevent distortion of the distal port 56 as the distal end 56 is slid over the adjustable basket 8 and the self-expanding thrombus elements 11a, 11b. In at least one embodiment, the delivery sheath 50 optionally includes a radiopaque band 58 positioned at the distal end 56 for identifying the position of the distal end 56 with fluoroscopy or other imaging techniques during a procedure. As illustrated in FIGS. 8A-D, the delivery sheath 50 is advanced distally over the intravascular catheter assembly 19 to fully sheath the adjustable basket 8 and the self-expanding thrombus elements 11a, 10b for delivery of the thrombectomy system 85 to and across the clot.

Following delivery and positioning of the intravascular catheter assembly 19 and the delivery sheath 50, the delivery sheath 50 is withdrawn proximally to unsheathe the adjustable basket 8. The unsheathed adjustable basket 8 is expanded radially to at least partially occlude the vessel by self-expansion, manual expansion by the movement of the inner catheter 15 as described above, or combination thereof. The delivery sheath 50 is optionally further withdrawn to unsheathe one or more self-expanding thrombus elements 11a, 11b, that expand radially to at least partially occlude the vessel. As illustrated in FIGS. 8A-D, in at least one embodiment, the outer catheter 17 optionally includes at least one marker band 59 denoting which baskets have been unsheathed and against which the proximal end 54 of the elongated sheath body 50 can be aligned to unsheathe the desired combination of baskets. The spacing of the baskets and the movement of the delivery sheath 50 allows an operator to select the number of baskets for deployment to treat a clot. As an example, depending on the location, the useable vessel length distal to the clot may be limited. Similarly, for shorter clots or smaller volumes, an operator may choose fewer baskets to minimize potential effects on the vessel wall. The thrombectomy system 85 is optionally used in conjunction with the thrombectomy guidewire 10 having the self-expanding basket 20, as shown in FIG. 2A-2B. For example, the thrombectomy guidewire 10 and self-expanding basket 20 are placed in the vessel and the thrombectomy system 85 is navigated along the thrombectomy guidewire 10 for deployment at a position proximal to the self-expanding basket 20. In this way, and as described herein, the self-expanding basket 20 catches or collects clot material that potentially breaks loose from the adjustable basket 8 or the self-expanding thrombus elements 11a, 11b.

As shown in FIGS. 9A-9B, the delivery sheath 50 optionally includes a hub assembly 68 having a primary branch 70 and a side branch 72. The primary branch 70 is axially aligned with the elongated sheath body 52 and defines a lumen extending the lumen of the elongated sheath body 52. The primary branch 70 includes a locking element 74 such as a locking screw or a friction lock for engaging the outer catheter 17 of the intravascular catheter assembly 19. The locking element 74 can be tightened to fix the delivery sheath 50 at a particular position axially along the outer catheter 17. The side branch 72 defines a lumen fluidly connected to the lumen of the primary branch 70. The side branch 72 includes a valve 76 and a port 78. The port 78 can be used to deliver fluid for flushing the delivery sheath 50 or applying vacuum for aspirating the delivery sheath 50, while the valve 76 can control fluid flow through the side branch 72.

As shown in FIGS. 9A-9B, in an embodiment, the thrombectomy system 85 includes a handle assembly 80 operably connected to the proximal end of the intravascular catheter assembly 19. The handle assembly 80 includes a handle body 82 and a translation assembly 84 for axially advancing and withdrawing the inner catheter 15 of the intravascular catheter assembly 19. Generally, the outer catheter 17 is fixedly attached to the handle body 82 while the translation assembly 84 is configured to advance and withdraw the inner catheter 15 relative to the outer catheter 17 to contract and expand the adjustable basket 8.

Intravascular devices usable with the wire shaft can include guidewire deliverable thrombectomy devices such as described in U.S. Pat. Nos. 9,827,084; 10,813,663; and U.S. Ser. No. 17/896,589. All of these cases are incorporated by reference herein.

### VARIOUS NOTES AND ASPECTS

Aspect 1 can include subject matter such as a thrombectomy guidewire system for delivering an intravascular thrombectomy device, comprising: a thrombectomy guidewire including: a wire shaft having a proximal end and a distal end, the wire shaft is sized for receiving and delivering guidewire deliverable intravascular thrombectomy device, and a self-expanding basket positioned at the distal end of the wire shaft and configured to self-expand from a collapsed configuration to an expanded configuration when constrained to press radially outward against vessel walls; and an exchange sheath defining a first lumen sized to slidably receive the wire shaft and a second lumen aligned with the central lumen; and wherein the exchange sheath is slidable in a distal direction over the wire shaft from the proximal end of the wire shaft at least until the self-expanding basket is sheathed within the central lumen and collapsed into the collapsed configuration.

Aspect 2 can include, or can optionally be combined with the subject matter of Aspect 1, to optionally include wherein the wire shaft has an outer diameter of at least one of about 0.014, about 0.018, and about 0.035 inches.

Aspect 3 can include, or can optionally be combined with the subject matter of one or any combination of Aspects 1 or 2 to optionally include wherein the wire shaft further comprises: a tapered wire tip positioned on the distal end of the wire shaft; wherein the wire tip tapers radially from a larger diameter proximal end of the dilator to a smaller diameter distal end of the dilator.

Aspect 4 can include, or can optionally be combined with the subject matter of one or any combination of Aspects 1-3 to optionally include wherein the outer diameter of the proximal end of the wire tip approximates the outer diameter of the guide sheath; wherein the guide sheath is slidable over the wire shaft in the distal direction to abut the distal end of the guide sheath against the proximal end of the wire tip.

Aspect 5 can include, or can optionally be combined with the subject matter of one or any combination of Aspects 1-4 to optionally include wherein the exchange sheath has a proximal end and a distal end; wherein the first lumen extends from the proximal end to the distal end of the exchange sheath.

Aspect 6 can include, or can optionally be combined with the subject matter of Aspects 1-5 to optionally include wherein the second lumen extends from the proximal end to the distal end of the exchange sheath.

Aspect 7 can include, or can optionally be combined with the subject matter of Aspects 1-6 to optionally include wherein the second lumen extends from a proximal opening in a sidewall of the guide sheath to a distal opening in the sidewall of the guide sheath; wherein the proximal opening and the distal opening are positioned between the proximal end and the distal end of the guide sheath.

Aspect 8 can include, or can optionally be combined with the subject matter of Aspects 1-7 to optionally include wherein the self-expanding basket has an outer diameter that is at least 30 mm when the self-expanding basket is in the expanded configuration.

Aspect 9 can include, or can optionally be combined with the subject matter of Aspects 1-8 to optionally include wherein the self-expanding basket has an outer diameter between about 32 mm to about 36 mm when the self-expanding basket is in the expanded configuration.

Aspect 10 can include, or can optionally be combined with subject matter of Aspects 1-9 to optionally include an adjustable basket and a sheath slidably coupled along the wire shaft and self-expanding thrombus elements slidably coupled along the wire shaft and proximal of the adjustable basket.

Aspect 11 can include, or can optionally be combined with the subject matter of Aspects 1-10 to optionally include wherein the self-expanding basket further comprises: a fenestrated structure extending between a proximal collar and a distal collar, the fenestrated structure defining a plurality of openings permitting fluid flow through the self-expanding basket while capturing solid material within the self-expanding basket; wherein the proximal collar is fixedly attached to the wire shaft and the distal collar is slidably attached to the wire shaft, wherein the distal collar slides distally along the wire shaft when the self-expanding basket is radially constrained to collapse the self-expanding basket and slides proximally along the wire shaft to expand the self-expanding basket.

Aspect 12 can include, or can optionally be combined with the subject matter of Aspects 1-11 to optionally include wherein the wire shaft further comprises: a wire stop limiting axial movement of the distal collar in the proximal direction to prevent radial expanding of the self-expanding basket beyond a first outer diameter.

Aspect 13 can include, or can optionally be combined with the subject matter of Aspects 1-12 to optionally include wherein the first outer diameter is about 36 mm.

Aspect 14 can include subject matter including a thrombectomy system for removing clot from a vessel, comprising: a thrombectomy guidewire including: a wire shaft having a proximal end and a distal end, and a self-expanding basket positioned at the distal end of the wire shaft and configured to self-expand from a collapsed configuration to an expanded configuration when unconstrained to provide embolic protection; an exchange sheath defining a central lumen sized to slidably receive the wire shaft and a secondary lumen oriented parallel to the central lumen, wherein the exchange sheath is slidable in a distal direction over the wire shaft from the proximal end of the wire shaft at least until the self-expanding basket is sheathed within the central lumen and collapsed into the collapsed configuration; and an intravascular thrombectomy device defining a device lumen sized to slidably receive the wire shaft such that the intravascular thrombectomy device is slidable over the wire shaft.

Aspect 15 can include, or can optionally be combined with the subject matter of Aspects 1-14 to optionally include wherein the wire shaft has an outer diameter of at least one of about 0.014, about 0.018, and about 0.035 inches.

Aspect 16 can include, or can optionally be combined with the subject matter of Aspects 1-15 to optionally include wherein the wire shaft further comprises: a tapered wire tip positioned on the distal end of the wire shaft; wherein the wire tip tapers radially from a larger diameter proximal end of the dilator to a smaller diameter distal end of the dilator.

Aspect 17 can include, or can optionally be combined with the subject matter of Aspects 1-16 to optionally include wherein the outer diameter of the proximal end of the wire tip approximates the outer diameter of the guide sheath; wherein the guide sheath is slidable over the wire shaft in the distal direction to abut the distal end of the guide sheath against the proximal end of the wire tip.

Aspect 18 can include, or can optionally be combined with the subject matter of Aspects 1-17 to optionally include wherein the exchange sheath has a proximal end and a distal end; wherein the central lumen extends from the proximal end to the distal end of the exchange sheath.

Aspect 19 can include, or can optionally be combined with the subject matter of Aspects 1-18 to optionally include wherein the secondary lumen extends from the proximal end to the distal end of the exchange sheath.

Aspect 20 can include, or can optionally be combined with the subject matter of Aspects 1-19 to optionally include wherein the secondary lumen extends from a proximal opening in a sidewall of the guide sheath to a distal opening in the sidewall of the guide sheath; wherein the proximal opening and the distal opening are positioned between the proximal end and the distal end of the guide sheath.

Aspect 21 can include, or can optionally be combined with the subject matter of Aspects 1-20 to optionally include wherein the self-expanding basket has an outer diameter that is at least 30 mm when the self-expanding basket is in the expanded configuration.

Aspect 22 can include, or can optionally be combined with the subject matter of Aspects 1-21 to optionally include wherein the self-expanding basket has an outer diameter between about 32 mm to about 36 mm when the self-expanding basket is in the expanded configuration.

Aspect 23 can include, or can optionally be combined with the subject matter of Aspects 1-22 to optionally include wherein the self-expanding basket further comprises: a fenestrated structure extending between a proximal collar and a distal collar, the fenestrated structure defining a plurality of openings permitting fluid flow through the self-expanding basket while capturing solid material within the self-expanding basket; wherein the proximal collar is fixedly attached to the wire shaft and the distal collar is slidably attached to the wire shaft, wherein the distal collar slides distally along the wire shaft when the self-expanding basket is radially constrained to collapse the self-expanding basket and slides proximally along the wire shaft to expand the self-expanding basket.

Aspect 24 can include, or can optionally be combined with the subject matter of Aspects 1-23 to optionally include wherein the wire shaft comprises: a wire stop limiting axial movement of the distal collar in the proximal direction to prevent radial expanding of the self-expanding basket beyond a first outer diameter.

Aspect 25 can include, or can optionally be combined with the subject matter of Aspects 1-24 to optionally include wherein the first outer diameter is about 36 mm.

Aspect 26 can include, or can optionally be combined with the subject matter of Aspects 1-25 to optionally include a procedural guidewire; wherein the secondary lumen of the exchange sheath is sized to slidably receive the procedural guidewire such that the exchange sheath is navigable over the procedural guidewire.

Aspect 27 can include, or can optionally be combined with the subject matter of Aspects 1-26 to optionally include a procedural sheath defining a procedural lumen sized to deliver at least the intravascular device through the vasculature.

Aspect 28 can include, or can optionally be combined with the subject matter of Aspects 1-27 to optionally include an adjustable basket and a sheath slidably coupled along the wire shaft and self-expanding thrombus elements slidably coupled along the wire shaft and proximal of the adjustable basket.

Aspect 29 can include, or can optionally be combined with the subject matter of Aspects 1-28 to optionally include a method of positioning downstream embolic protection during thrombectomy procedure comprising: advancing an exchange sheath over a wire shaft of a thrombectomy guidewire, wherein the wire shaft is received within a central lumen of the exchange sheath; sliding the exchange sheath distally over a self-expanding basket on a distal end of the wire shaft to collapse the self-expanding basket to a collapsed configuration; navigating a procedural guidewire through a vessel containing a thrombus such that a distal end of the procedural guidewire is positioned distal to the clot; advancing the guide sheath with the thrombectomy guidewire over the procedural guidewire until the self-expanding basket is positioned distal to the clot, wherein the procedural guidewire is received within a secondary lumen of the exchange sheath; withdrawing the procedural guidewire from the vessel through the secondary lumen of the exchange sheath; and withdrawing the exchange sheath over the wire shaft to unsheathe the self-expanding basket and permit the self-expanding basket to expand within the vessel distal to the clot.

Aspect 30 can include, or can optionally be combined with the subject matter of Aspects 1-29 to optionally include advancing a procedural sheath over the procedural guidewire such that a proximate end of the procedural sheath is proximate to the clot.

Aspect 31 can include, or can optionally be combined with the subject matter of Aspects 1-30 to optionally include advancing an intravascular device over the wire sheath to the clot; and engaging the clot with the intravascular device.

Aspect 32 can include, or can optionally be combined with the subject matter of Aspects 1-31 to optionally include wherein the self-expanding basket prevents emboli separating from the clot during engagement by the intravascular device from passing through the self-expanding basket.

Aspect 33 can include, or can optionally be combined with the subject matter of Aspects 1-32 to optionally include sliding the exchange sheath distally over an adjustable basket and a sheath, the sheath slidably coupled along the wire shaft and over self-expanding thrombus elements slidably coupled along the wire shaft and proximal of the adjustable basket and withdrawing the exchange sheath over the wire shaft to unsheathe the adjustable basket and the self-expanding thrombus elements.

Each of these non-limiting aspects can stand on its own, or can be combined in various permutations or combinations with one or more of the other aspects.

The above description includes references to the accompanying drawings, which form a part of the detailed description. The drawings show, by way of illustration, specific embodiments in which the subject matter can be practiced. These embodiments are also referred to herein as “aspects” or “examples.” Such aspects or example can include elements in addition to those shown or described. However, the present inventors also contemplate aspects or examples in which only those elements shown or described are provided. Moreover, the present inventors also contemplate aspects or examples using any combination or permutation of those elements shown or described (or one or more features thereof), either with respect to a particular aspects or examples (or one or more features thereof), or with respect to other Aspects (or one or more features thereof) shown or described herein.

In the event of inconsistent usages between this document and any documents so incorporated by reference, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

Geometric terms, such as “parallel”, “perpendicular”, “round”, or “square”, are not intended to require absolute mathematical precision, unless the context indicates otherwise. Instead, such geometric terms allow for variations due to manufacturing or equivalent functions. For example, if an element is described as “round” or “generally round,” a component that is not precisely circular (e.g., one that is slightly oblong or is a many-sided polygon) is still encompassed by this description.

The above description is intended to be illustrative, and not restrictive. For example, the above-described aspects or examples (or one or more aspects thereof) may be used in combination with each other. Other embodiments can be used, such as by one of ordinary skill in the art upon reviewing the above description. The Abstract is provided to comply with 37 C.F.R. § 1.72(b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as aspects, examples or embodiments, with each claim standing on its own as a separate embodiment, and it is contemplated that such embodiments can be combined with each other in various combinations or permutations. The scope of the subject matter should be determined with reference to the appended claims, along with the full scope of equivalents to which such claims are entitled.

1. A thrombectomy guidewire system for delivering an intravascular thrombectomy device, comprising:
a thrombectomy guidewire including:
a wire shaft having a proximal end and a distal end, the wire shaft is sized for receiving and delivering a guidewire deliverable intravascular thrombectomy device; and
a self-expanding basket positioned at the distal end of the wire shaft and configured to self-expand from a collapsed configuration to an expanded configuration to press radially outward against vessel walls;
an exchange sheath defining a first lumen configured to slidably receive the wire shaft and a second lumen laterally offset from the first lumen; and
wherein the exchange sheath is slidable in a distal direction over the wire shaft from the proximal end of the wire shaft at least until the self-expanding basket is sheathed within the first lumen and collapsed into the collapsed configuration.

2. The thrombectomy guidewire system of claim 1, wherein the wire shaft has an outer diameter of at least one of about 0.014, about 0.018, and about 0.035 inches.

3. The thrombectomy guidewire system of claim 1, wherein the wire shaft further comprises:
a tapered wire tip positioned on the distal end of the wire shaft;
wherein the wire tip tapers radially from a larger diameter proximal end of the dilator to a smaller diameter distal end of the dilator.

4. The thrombectomy guidewire system of claim 3, wherein an outer diameter of the proximal end of the wire tip approximates an outer diameter of the exchange sheath;
wherein the exchange sheath is slidable over the wire shaft in the distal direction to abut the distal end of the guide sheath against the proximal end of the wire tip.

5. The thrombectomy guidewire system of claim 1, wherein the exchange sheath has a proximal end and a distal end;
wherein the first lumen extends from the proximal end to the distal end of the exchange sheath.

6. The thrombectomy guidewire system of claim 5, wherein the second lumen extends from the proximal end to the distal end of the exchange sheath.

7. The thrombectomy guidewire system of claim 5, wherein the second lumen extends from a proximal opening in a sidewall of the exchange sheath to a distal opening in the sidewall of the exchange sheath,
wherein the proximal opening and the distal opening are positioned between the proximal end and the distal end of the exchange sheath.

8. The thrombectomy guidewire system of claim 1, wherein the self-expanding basket has an outer diameter that is at least 30 mm when the self-expanding basket is in the expanded configuration.

9. The thrombectomy guidewire system of claim 1, wherein the self-expanding basket has an outer diameter between about 32 mm to about 36 mm when the self-expanding basket is in the expanded configuration.

10. The thrombectomy guidewire system of claim 1, further comprising:
an adjustable basket and a sheath slidably coupled along the wire shaft; and
self-expanding thrombus elements slidably coupled along the wire shaft and proximal of the adjustable basket.

11. The thrombectomy guidewire system of claim 1, wherein the self-expanding basket further comprises:
a fenestrated structure extending between a proximal collar and a distal collar, the fenestrated structure defining a plurality of openings permitting fluid flow through the self-expanding basket while capturing solid material within the self-expanding basket;
wherein the proximal collar is fixedly attached to the wire shaft and the distal collar is slidably attached to the wire shaft, wherein the distal collar slides distally along the wire shaft when the self-expanding basket is radially constrained to collapse the self-expanding basket and slides proximally along the wire shaft to expand the self-expanding basket.

12. The thrombectomy guidewire system of claim 11, wherein the wire shaft comprises:
a wire stop limiting axial movement of the distal collar in the proximal direction to prevent radial expanding of the self-expanding basket beyond a first outer diameter.

13. The thrombectomy guidewire system of claim 12, wherein the first outer diameter is about 36 mm.

14. A thrombectomy system for removing clot from a vessel, comprising:
a thrombectomy guidewire including:
a wire shaft having a proximal end and a distal end; and
a self-expanding basket positioned at the distal end of the wire shaft and configured to self-expand from a collapsed configuration to an expanded configuration when unconstrained to provide embolic protection;
a exchange sheath defining a central lumen sized to slidably receive the wire shaft and a secondary lumen oriented parallel to the central lumen, wherein the exchange sheath is slidable in a distal direction over the wire shaft from the proximal end of the wire shaft at least until the self-expanding basket is sheathed within the central lumen and collapsed into the collapsed configuration; and
an intravascular thrombectomy device having a device lumen configured to slidably receive the wire shaft, the intravascular thrombectomy device is slidable over the wire shaft.

15. The thrombectomy system of claim 14, wherein the wire shaft has an outer diameter of at least one of about 0.014, about 0.018, and about 0.035 inches.

16. The thrombectomy system of claim 14, wherein the wire shaft further comprises:
a tapered wire tip positioned on the distal end of the wire shaft;
wherein wire tip tapers radially from a larger diameter proximal end of the dilator to a smaller diameter distal end of the dilator.

17. The thrombectomy system of claim 16, wherein an outer diameter of the proximal end of the wire tip approximates the outer diameter of the exchange sheath;
wherein the exchange sheath is slidable over the wire shaft in the distal direction to abut the distal end of the guide sheath against the proximal end of the wire tip.

18. The thrombectomy system of claim 14, wherein the exchange sheath has a proximal end and a distal end;
wherein the central lumen extends from the proximal end to the distal end of the exchange sheath.

19. The thrombectomy system of claim 18, wherein the secondary lumen extends from the proximal end to the distal end of the exchange sheath.

20. The thrombectomy system of claim 18, wherein the secondary lumen extends from a proximal opening in a sidewall of the guide sheath to a distal opening in the sidewall of the exchange sheath,
wherein the proximal opening and the distal opening are positioned between the proximal end and the distal end of the exchange sheath.

21. The thrombectomy system of claim 14, wherein the self-expanding basket has an outer diameter that is at least 30 mm when the self-expanding basket is in the expanded configuration.

22. The thrombectomy system of claim 14, wherein the self-expanding basket has an outer diameter between about 32 mm to about 36 mm when the self-expanding basket is in the expanded configuration.

23. The thrombectomy system of claim 14, wherein the self-expanding basket further comprises:
a fenestrated structure extending between a proximal collar and a distal collar, the fenestrated structure defining a plurality of openings permitting fluid flow through the self-expanding basket while capturing solid material within the self-expanding basket;
wherein the proximal collar is fixedly attached to the wire shaft and the distal collar is slidably attached to the wire shaft, wherein the distal collar slides distally along the wire shaft when the self-expanding basket is radially constrained to collapse the self-expanding basket and slides proximally along the wire shaft to expand the self-expanding basket.

24. The thrombectomy guidewire system of claim 23, wherein the wire shaft further comprises:
a wire stop limiting axial movement of the distal collar in the proximal direction to prevent radial expanding of the self-expanding basket beyond a first outer diameter.

25. The thrombectomy guidewire system of claim 24, wherein the first outer diameter is about 36 mm.

26. The thrombectomy guidewire system of claim 14, further comprising:
a procedural guidewire;
wherein the secondary lumen of the exchange sheath is sized to slidably receive the procedural guidewire such that the exchange sheath is navigable over the procedural guidewire.

27. The thrombectomy guidewire system of claim 26, further comprising:
a procedural sheath defining a procedural lumen sized to deliver at least the intravascular device through the vessel.

28. The thrombectomy guidewire system of claim 14, further comprising:
an adjustable basket and a sheath slidably coupled along the wire shaft; and
self-expanding thrombus elements slidably coupled along the wire shaft and proximal of the adjustable basket.

29. A method of positioning downstream embolic protection during thrombectomy procedure comprising:
advancing an exchange sheath over a wire shaft of a thrombectomy guidewire, wherein the wire shaft is received within a central lumen of the exchange sheath;
sliding the exchange sheath distally over a self-expanding basket on a distal end of the wire shaft to collapse the self-expanding basket to a collapsed configuration;
navigating a procedural guidewire through a vessel containing a thrombus such that a distal end of the procedural guidewire is positioned distal to a clot;
advancing the exchange sheath with the thrombectomy guidewire over the procedural guidewire until the self-expanding basket is positioned distal to the clot, wherein the procedural guidewire is received within a secondary lumen of the exchange sheath;
withdrawing the procedural guidewire from the vessel through the secondary lumen of the exchange sheath; and
withdrawing the exchange sheath over the wire shaft to unsheathe the self-expanding basket and permit the self-expanding basket to expand within the vessel distal to the clot.

30. The method of claim 29, further comprising:
advancing a procedural sheath over the procedural guidewire such that a proximate end of the procedural sheath is proximate to the clot.

31. The method of claim 30 further comprising:
advancing an intravascular device over the wire sheath to the clot; and
engaging the clot with the intravascular device.

32. The method of claim 31, wherein the self-expanding basket prevents emboli separating from the clot during engagement by the intravascular device from passing through the self-expanding basket.

33. The method of claim 29, further comprising:
sliding the exchange sheath distally over an adjustable basket and a sheath, the sheath slidably coupled along the wire shaft and over self-expanding thrombus elements slidably coupled along the wire shaft and proximal of the adjustable basket; and
withdrawing the exchange sheath over the wire shaft to unsheathe the adjustable basket and the self-expanding thrombus elements.
